(19) United States
(12) Patent Application Publication
(10) Pub. No.: US 2004/0167441 A1
(43) Pub. Date: Aug. 26, 2004

(54) Composite medical device

(76) Inventors: Brian R. Reynolds, Ramsey, MN (US); Peter Skujins, Minneapolis, MN (US)

Correspondence Address: J. Scot Wickhem, CROMPTON, SEAGER & TUFTE, LLC, Suite 895, 331 Second Avenue South, Minneapolis, MN, 55401-2246, US

(21) Appl. No.: 10/375,766

(22) Filed: Feb. 26, 2003

(51) Int. Cl. A61B 5/00

(52) U.S. Cl. 600/585

(57) ABSTRACT

Alternative designs, materials and manufacturing methods for medical devices. Some embodiments pertain to a composite medical device including an first section having an end, and a connector member defining an opening therein, wherein the end of the elongated section extends into the opening. A bismuth alloy connector material is disposed within the opening, and the connector material is configured to expand when solidified to exert a compressive force within the connector member.

### FIELD OF THE INVENTION

The invention generally pertains to medical devices, and more specifically to composite medical devices including two or more structural elements connected together, and a method of making the same.

### BACKGROUND

A wide variety of medical devices, such as guidewires, catheters, and the like, have been developed for use in facilitating navigation and treatment throughout the anatomy of a patient. Because the anatomy of a patient may be very tortuous, it is desirable to combine a number of performance features in such a medical device. It is generally known to provide medical devices including multiple structural elements connected together to provide a number of performance features in a medical device. The prior art offers a number of different structures and mechanisms for connecting structural elements in medical devices. Each of these different structures and mechanisms has certain advantages and disadvantages. However, there is an ongoing need to provide alternative medical device structures and assemblies.

### SUMMARY

The invention provides several alternative designs, materials and methods of manufacturing alternative medical device structures and assemblies.

### BRIEF DESCRIPTION OF THE DRAWINGS

The invention may be more completely understood in consideration of the following detailed description of various embodiments of the invention in connection with the accompanying drawings, in which:

FIG. 1 is cross sectional fragmentary view of a medical device shown as a guidewire (pre-grinding), including a connection utilizing an overlapping tapered joint and a tubular connector for joining a proximal section and a distal section of the guidewire;

FIG. 2 is a cross sectional fragmentary view of the guidewire (post grinding) of FIG. 1;

FIG. 3 is a cross sectional fragmentary view of an alternative guidewire (post grinding), including a connection utilizing a butt joint and a tubular connector for joining a proximal section and a distal section of the guide wire;

FIG. 4 is a cross sectional fragmentary view of an alternative guidewire (post grinding), including a connection utilizing an overlapping joint and a tubular connector for joining a proximal section and a distal section of the guide wire;

FIGS. 5A-5C are cross sectional fragmentary views of various end portions for use with the guidewire embodiment of FIG. 4; and

FIG. 6 is a is a cross sectional fragmentary view of an alternative guidewire, including a connection utilizing a joint and a tubular connector for joining a proximal section and a distal section of the guide wire, wherein there is spacing between the the proximal section and the distal section of the guide wire.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The detailed description and drawings illustrate examples of various embodiments of the claimed invention, and are not intended to be limiting.

At least some embodiments of the invention provide a medical device, or components or structures for use in a medical device, that include two or more structural elements that are connected using a bismuth alloy connector material. The bismuth alloy connector material is configured to expand when solidified. Some embodiments include a structural member defining an opening therein, and another component or structure that includes a portion that extends into the opening. The bismuth alloy connector material is also present within the opening, and upon solidification, the bismuth alloy connector material expands to exert a compressive force within the opening. The compressive force acts to connect the structural member to the other component or structure by exerting the compressive force on the inner surface of the opening of the structural member and on the portion of the other component or structure that extends into the opening. A mechanical interlock is thereby provided between the structural member and the other component or structure.

In some embodiments, the use of such a bismuth alloy connector material can provide for some advantages. For example, in some embodiments, because the connection provided by the using such material does not depend on alloying with, or chemically adhering to the materials of the components being connected, there is less of a concern regarding providing surfaces to be attached that are absolutely free of contaminants or surface oxides. Thus, where conventional preparation of surfaces of some materials that are to be connected may require a substantial amount of treatment or preparation, many such treatments or preparations are not necessary in at least some embodiments using a bismuth alloy connector material. This can be advantageous for many reasons, especially in situations the components to be connected are difficult to expose to such treatments. For example, some components may be very small, or may include portions that are physically difficult to reach with such treatments or preparations. Additionally, some components may be made of materials that do not react well with, or cannot be exposed to harsh preparation or treatment methods.

In some embodiments, the structural member is a connector member that is particularly adapted and configured to interconnect two or more other components or structures. For example, the structural member may be a connector tube that is particularly adapted and configured to have portions of other structures, such as the ends of elongated members such as wires or hypotubes, inserted therein for connection using the bismuth alloy connector material. In some other embodiments, the structural member is a component of the medical device that includes an opening or aperture on a portion thereof that is adapted and configured for use in connecting the structural member with other components of the device using the bismuth alloy connector material. For example, the structural member may be a wire or hypotube defining an opening or lumen in the end thereof, or other such structure.

Although discussed with specific reference to guidewires in much of the description below, the invention may be applicable to almost any medical device. For example, the invention may be applicable to shafts for catheters (e.g., guide catheters, balloon catheters, stent delivery catheters, etc.), infusion devices, distal protection devices, or shafts for rotational devices (atherectomy catheters, IVUS catheters, etc.). In some embodiments, the medical device is particularly adapted and configured for use in intravascular applications.

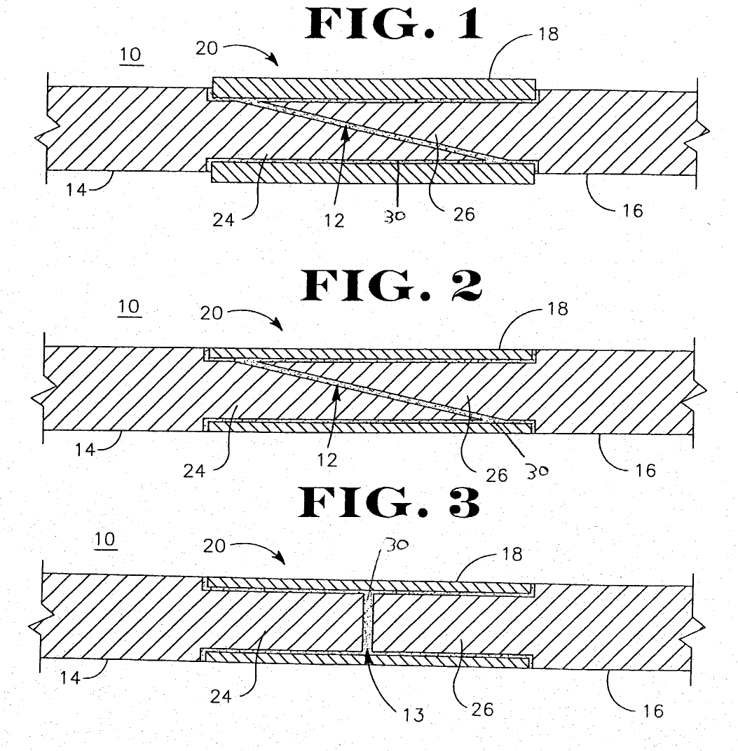

Refer now to FIGS. 1-4 which illustrate cross sectional views of a portion of a guidewire 10 including a connection 20 joining a proximal guidewire section 14 and a distal guidewire section 16. FIG. 1 illustrates the guidewire 10 and the connection 20 before a final grinding step, and FIG. 2 illustrates the guidewire 10 and the connection 20 after the final grinding step, which provides a smooth outer profile. The embodiment of FIGS. 1 and 2 utilizes an overlapping tapered joint 12, a connector structure 18 and a bismuth alloy connector material 30.

The embodiment of FIG. 3 is similar to the embodiment of FIGS. 1 and 2, except that the connection 20 between the proximal guidewire section 14 and the distal 5 guidewire section 16 does not utilize an overlapping joint 12, but rather uses a butt joint 13. The embodiment of FIG. 4 is also similar to the embodiment of FIGS. 1 and 2, except that the connection 20 between the proximal guidewire section 14 and the distal guidewire section 16 utilizes an overlapping joint 12 that is not tapered.

Those of skill in the art and others will recognize that the materials, structure, and dimensions of the proximal/distal guidewire sections 14/16 are dictated primary by the desired characteristics and function of the final guidewire, and that any of a broad range of materials, structures, and dimensions can be used.

For example, the proximal and distal guidewire sections 14/16 may have a solid cross-section as shown, or a hollow cross-section, and may be formed of any materials suitable for use, dependent upon the desired properties of the guidewire. Some examples of suitable materials include metals, metal alloys, and polymers. In some embodiments, it is desirable to use metals, or metal alloys that are suitable for metal joining techniques such as welding, soldering, brazing, crimping, friction fitting, adhesive bonding, etc. As used herein, the proximal section 14 and the distal section 16 may generically refer to any two adjacent guidewire sections along any portion of the guidewire.

In some embodiments, the proximal guidewire section 14 may be formed of relatively stiff material such as straightened 304v stainless steel wire. Alternatively, proximal portion 14 may be comprised of a metal or metal alloy such as a nickel-titanium alloy, nickel-chromium alloy, nickel-chromium-iron alloy, cobalt alloy, a polymer material, such as a high performance polymer, or other suitable material, or the like. In general, the material used to construct proximal portion 14 may be selected to be relatively stiff for pushability and torqueability.

In some embodiments, the distal guidewire section 16 may be formed of a relatively flexible material such as a straightened super elastic (i.e. pseudoelastic) or linear elastic alloy (e.g., nickel-titanium) wire, or a alternatively, a polymer material, such as a high performance polymer, or similar such material or the like. Alternatively, distal portion 16 may be comprised of a metal or metal alloy such as stainless steel, nickel-chromium alloy, nickel-chromium-iron alloy, cobalt alloy, or other suitable material. In general, the material used to construct distal portion 16 may be selected to be relatively flexible for trackability.

In some particular embodiments, the distal section 16 is a linear elastic nickel-titanium alloy, for example, linear elastic nitinol. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

Within the family of commercially available nitinol alloys, is a category designated “linear elastic” which, although is similar in chemistry to conventional shape memory and superelastic varieties, exhibits distinct and useful mechanical properties. By skilled applications of cold work, directional stress, and heat treatment, the wire is fabricated in such a way that it does not display a substantial “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range. For example, in some embodiments, there is no martensite/austenite phase changes detectable by DSC and DMTA analysis in the range of about −60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy for the distal portion 16 allows the guidewire to exhibit superior “pushability” around tortuous anatomy.

In some embodiments, the linear elastic nickel-titanium alloy comprises in the range of about 50 to about 60 wt. % nickel, with the remainder being essentially titanium. In some particular embodiments, the composition comprises in the range of about 54 to about 57 wt. % nickel. One example of a suitable nickel-titanium alloy is FHP-NT alloy commercially available from Furukawa Techno Material Co. of Kanagawa, Japan. Some examples of nickel-titanium alloys are disclosed in U.S. Pat. Nos. 5,238,004 and 6,508,803, which are herein incorporated by reference.

In some particular embodiments, the proximal guidewire section 14 is formed from a stainless steel wire having a diameter in the range of 0.01 to 0.02 inches, and a length in the range of about 50 to about 110 inches, and the distal guidewire section 16 is formed from a linear elastic nitinol wire having a diameter that ranges from a diameter to match the diameter of the proximal guidewire section 14 to as small as about 0.002 inches, and a length in the range of 3 to 15 inches.

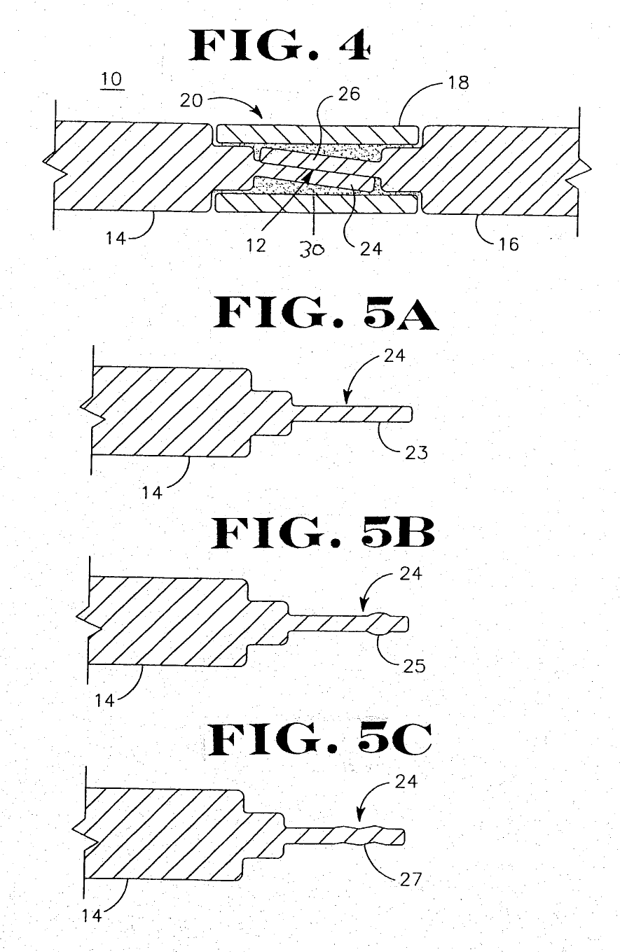

The distal end 24 of the proximal portion 14 and the proximal end 26 of distal portion 16 (i.e., the joined ends) may form an overlapping tapered joint 12 as shown in FIGS. 1-2. Alternatively, the joined ends 24/26 may form a butt joint 13 as shown in FIG. 3. The joined ends 24/26 in the a butt joint 13 can be in direct contact with each other, or can include a degree of spacing between the joined ends, as shown in FIG. 3. As a further alternative, the joined ends 24/26 may form an overlapping joint 12 that is not tapered as shown in FIG. 4. The one or both of the non-tapered end portions 24/26 may have a uniform profile (diameter) 23 as shown in FIG. 5A, a bulbous portion 25 for purposes of mechanical interlocking as shown in FIG. 5B, or a helical form 27 for purposes of mechanical interlocking as shown in FIG. 5C. Additionally, in some embodiments, using either an overlapping type joint or using a butt type joint, portions of the outer surfaces of either of the joined ends can be provided with additional structures, such as grooves, ridges, a roughened or textured surface, or the like for the purpose of providing better mechanical interlocking between the joined ends and the connector structure or connector material.

In each of the embodiments illustrated in FIGS. 1-2 and 4, the end portions 24/26 overlap to form an overlapping joint 12. The overlapping joint 12 can act to blend the stiffness of proximal portion 14 and distal portion 16, if desired, by combining the properties of each end section 24/26 making up the cross section of the overlapping joint 12. Thus, the joint 12 forms a flexibility transition region that has a relative flexibility that is between the flexibility of the proximal portion 14 and the flexibility of the distal portion 16.

In the tapered embodiments illustrated in FIGS. 1-2, the ends 24/26 may be tapered or otherwise formed to have a mating geometry that gradually decreases in cross sectional area toward the middle of the connection 20. The tapered overlapping portion 12 may define a uniform or a non-uniform transition of the sections 24/26, depending on the transition characteristics desired. For example, the end sections 24/26 may be linearly tapered as shown, tapered in a curvilinear fashion, or tapered in a step-wise fashion. If tapered linearly as shown, the angle of the taper may vary. Using the longitudinal center axis of the guidewire 10 as a reference, as measured from the extreme ends of the end sections 24/26, the angle of the taper is acute (i.e., less than 90 degrees), and may be in the range of 5 degrees to 45 degrees, for example. Varying the angle of the tapered ends 24/26 also varies the length of the overlapping joint 12 in accordance with geometric principles. The length of the overlapping joint 12 may be selected to obtain a more (longer length) or less (shorter length) gradual transition in stiffness.

As mentioned previously, the proximal guidewire section 14 and the distal guidewire section 16 may be formed of different materials (i.e., materials having different moduli of elasticity) resulting in a difference in flexibility. For example, the proximal guidewire section 14 may be formed of stainless steel wire and the distal guidewire section 16 may be formed of nickel-titanium alloy wire, both having the same dimensions, resulting in a 3:1 difference in elastic modulus. Such a difference in elastic modulus (i.e., flexibility) may result in a stress concentration point during flexure and/or torsion that may have a tendency to kink and fracture. By virtue of the gradual transition in stiffness provided by the overlapping portion 12, stress is distributed along the entire length of the connection 20 thereby decreasing the probability that guidewire 10 may kink at the junction.

A gradual transition in stiffness may also allow the connection 20 to be located further distally. According to this embodiment, the distal portion 16 may be manufactured to be shorter than proximal portion 14. Including a relatively long proximal section 14 may advantageously increase the torquability and pushability of the guidewire 10. Although only one connection 20 is shown, additional connections 20 may be used to connect other guidewire sections of varying stiffness.

The connector structure 18 may comprise a structure defining one or more openings therein or one or more lumens extending there through. In some embodiments, the connector structure 18 is a generally tubular structure such as a hypotube as shown, or a coiled wire, or the like. The connector 18 may have an inside diameter sized and shaped appropriately to receive the ends 24/26 of the proximal portion 14 and the distal portion 16, and an outside diameter sufficient to accommodate a final grinding procedure. In the embodiment shown, the outside surface of the connector structure is generally circular in cross-sectional shape, however, other geometries, for example, oval, or multisided geometries may be used in other embodiments. In some embodiments, the connector 18 can include one or more grooves, slits, slots, or the like, that are defined in the body of the connector, for example, to provide a desired degree of flexability characteristics to the connector 18. Some examples of such structures are disclosed in a U.S. Patent Application entitled “ARTICULATING INTRACORPORAL MEDICAL DEVICE” (Attorney docket no. 1001.1668101) filed on even date herewith, which is incorporated herein by reference. Some other examples of suitable techniques and structures that can be used to interconnect different shaft sections are disclosed in U.S. patent application Ser. No. 09/972,276 filed on Oct. 5, 2001 and 10/068,992 filed on Feb. 28, 2002, which are incorporated herein by reference. Some additional examples of structures and materials that can be used in medical device constructions are also disclosed in a U.S. Patent Application entitled “ELONGATED INTRACORPORAL MEDICAL DEVICE” (Attorney docket no. 1001.1673101) filed on even date herewith, which is incorporated herein by reference.

In some example embodiments, the connector 18 is generally tubular, and can have an inner diameter in the range of about 0.005 to about 0.02 inches, and an outer diameter in the range of about 0.01 to about 0.025 inches. In some particular embodiments, the connector 18 can have and inner diameter of about 0.010 inches and an outer diameter of about 0.014 inches. The final diameter of the guidewire 10 and the connector 18 may be in the range of 0.010 to 0.018 inches, for example. By way of example, not limitation, the connector 18 may have a length of about 1.0 to 3.0 inches for an overlapping portion 12 of about 0.25 to 2.5 inches. However, in some other embodiments, this type of construction can be applied to wires or other structures of larger diameter intended, for example, for peripheral intervention purposes. Such wires could range as large as 0.035 in diameter and therefore have an extended length connector and correspondingly longer overlapping sections.

The connector 18 may include or be made of a metal or metal alloy, and may include radiopaque materials. Suitable metals and metal alloys include stainless steels, nickel-titanium alloys (e.g., nitinol), nickel-chromium alloys, nickel-chromium-iron alloys, cobalt alloys, nickel, or other suitable materials, and the like. An example is a nickel-chromium-iron alloy designated UNS N06625 and is available under the trade name INCONEL 625, which may be obtained from California Fine Wire Company of Grover Beach, Calif. One example of a nickel-molybdenum-chromium alloy is designated UNS 10276 and is available under the trade name ALLOY C276 from Fort Wayne Metals Research Products Corporation of Fort Wayne, Ind. An example of a nickel-molybdenum alloy are those of the of the Hastelloy family and an example of which is available under the trade name ALLOY B2 from Fort Wayne Metals Research Products Corporation of Fort Wayne, Ind. Alternatively, connector 18 may be comprised of a polymer or a metal-polymer composite, and the like.

As indicated above, the connector 18, or portions thereof can also be made of or include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, plastic material loaded with a radiopaque filler, and the like.

It should also be understood that in some embodiments, the connector structure, or the structure defining one or more openings therein, may be part of, or integral with, one of the structural elements being connected together. For example, the connector structure could be defined by an end of a wire or hypotube that includes an opening, recess, or lumen defined therein, into which the bismuth alloy connector material and the end of another structure are inserted for connection thereto. Additionally, the connector structure may be attached to one of the structures being connected together using more conventional techniques, such as adhesive bonding, thermal bonding, soldering, brazing, welding, mechanical connection (e.g. crimping, friction fitting, etc.), while being connected to another structures using the bismuth alloy connector material as discussed herein.

The bismuth alloy connector materials, for example connector material 30, for use in making the connection are adapted and configured to have the characteristic of expansion or growth upon or after solidification. In at least some embodiments, although normal thermal shrinkage of the liquid bismuth alloy may take place during cooling of the liquid, the crystalline structure that forms during solidification occupies a larger volume than the same mass of liquid. When the bismuth alloy connector materials are confined within a predetermined space, for example within an opening in a connector structure, compressive forces are generated as the alloy solidifies and expands or grows within the space.

Some examples of suitable bismuth alloy connector materials include alloys of bismuth including additional alloying elements such as tin, indium, cadmium, lead, and the like. Due to the fact that the final medical device will make contact with or be inserted into a living body, in some embodiments, the alloy should include only elements that are known to be acceptable for contact with the body. For example, bismuth alloys including elements such as tin, indium, or the like, may be more acceptable for contact with the body. Some example alloys can include in the range of about 4 to about 80 wt. % bismuth, with the remainder being other alloying elements. Some examples of suitable alloys, and example ranges of wt. % of components within some alloys, and some specific examples of such alloys, include those having the ranges of components as illustrated in Table 1 as follows:

TABLE 1

| | | One example of |
|---|---|---|
| | Range of | a specific alloy |
| | components in | falling within |
| Type of alloy: | some embodiments: | these ranges: |
| Bismuth-Tin alloy | 35 to 45 wt. % Bi, and | 40 wt. |
| | 55 to 65 wt. % Sn | 60 wt. % Sn |
| Bismuth-Tin alloy | 53 to 63 wt. % Bi, and | 58 wt. % Bi, and |
| | 37 to 47 wt. % Sn | 42 wt. % Sn |
| Bismuth-Indium alloy | 2 to 10 wt. % Bi, and | 5 wt. % Bi, and |
| | 90 to 98 wt. % In | 95 wt. % In |
| Bismuth-Indium alloy | 62 to 72 wt. % Bi, and | 67 wt. % Bi, and |
| | 28 to 38 wt. % In | 33 wt. % In |
| Bismuth-Indium alloy | 29 to 39% Bi, and | 34 wt. % Bi, and |
| | 61 to 71 wt. % In | 66 wt. % In |
| Bismuth-Indium-Tin | 53 to 63 wt. % Bi, | wt. % Bi, |
| alloy | 20 to 30 wt. % In, and | wt. % In, and |
| | 12 to 22 wt. % Sn | wt. % Sn |

At least some of the bismuth alloy that can be used as the connector material are characterized by relatively low melting temperatures compared to some other metal alloys. For example, in some embodiments, the bismuth alloy used is characterized as being a “fusible” alloy, meaning that it has a melting point in the range of about 50 to about 260 C. In some embodiments, the alloy has a melting point in the range of about 200 C or below, or in the range of about 150 C or below. In some embodiments, the alloy used is the eutectic alloy for the particular type of alloy being used, meaning that it is the particular alloy having the lowest melting point (i.e. eutectic point) that is obtainable by varying the proportions of the components of the alloy. Eutectic alloys have definite and minimum melting points in contrast to other combinations of the same metals. For such eutectic alloys, the minimum melting points as given above would be the eutectic melting point.

The low melting temperatures can be useful when the connector material is used in conjunction with structural elements that are-made of or include temperature sensitive material. For example, some nickel-titanium alloys are annealed or shape set by exposure to higher temperatures. Therefore, the use of alloys having a lower temperature melting point can help to preserve the desired heat-treat state of structures made of such nickel-titanium alloys that need to be connected to other structures.

To manufacture the connection 20 of the guidewire 10, the ends 24/26 of the proximal and distal guidewire sections 14/16 may be ground or otherwise worked to form the desired size and shape (e.g., uniform diameter 23, bulbous portion 25, helix 27, taper, or mechanical interlocking features, such as grooves, ridges, roughened surfaces, etc.) to accommodate the size and shape of the connector structure 18, or to accommodate the type of joint being constructed (e.g. an overlapping joint, a butt joint, etc.). Additionally, the size and shape of the ends 24/26 and the connector structure 18 can be configured to allow for the inclusion of the bismuth alloy connector material 30, and for the flow of the connector material in a liquid state. A recess step may be ground into the proximal and distal guidewire sections 14/16 to accommodate the connector structure, such as the connector tube 18.

The ends 24/26 of the proximal and distal guidewire sections 14/16 and the bismuth alloy connector material 30 are disposed within the lumen defined in the connector structure 18 in such a manner that the bismuth alloy connector material 30 solidifies and expands to exert a compressive force within the lumen. The compressive force within the lumen acts to connect the connector structure to the 18 ends 24/26 of the proximal and distal guidewire sections 14/16, and thereby connect the proximal and distal guidewire sections 14/16 to each other. The ends 24/26 of the proximal and distal guidewire sections 14/16 and the bismuth alloy connector material 30 can be disposed within the lumen of the connector structure 18 using any suitable process or method that allows for such a connection to be formed.

For example, in some embodiments, a sufficient amount of the bismuth alloy connector material is applied to either one or both ends 24/26 of the proximal and distal guidewire sections 14/16, and bismuth alloy connector material is allowed to solidify. The bismuth alloy connector material can be applied to the ends 24/26 using any suitable process, for example, a hot dipping process, a coating process, a spraying process, a plating process, or the like. The ends 24/26 are then inserted into the lumen within the connector tube 18 until a dimensional interference is created. The bismuth alloy connector material is then heated above its melting point of the bismuth alloy connector material, and an additional insertion force is applied to the guidewire sections 14/16 to provide axial movement of the ends 24/26 further into the connector structure and into a bonding position. For example, the distal end 24 of the proximal portion 14 and proximal end 26 of the distal portion 16 can be positioned adjacent one another in an overlapping 12 or an end-to-end 13 arrangement within the connector structure 18. In some embodiments, ends 24/26 are moved into the bonding position, some excess bismuth alloy connector material may be displaced from within the lumen in the connector structure 18, indicating that the opening or lumen if full to capacity. The bismuth alloy connector material 30 is allowed to cool and solidify within the lumen. As the bismuth alloy connector material solidifies, it expands to exert a compressive force within the lumen. The compressive force within the lumen acts to provide a mechanical interlock between the connector structure 18 to the ends 24/26 of the proximal and distal guidewire sections 14/16, and thereby connect the proximal and distal guidewire sections 14/16 to each other.

Additionally, in some embodiments, as a result of the bismuth alloy expansion within the lumen, there may be a slight amount of outward motion of the guidewire sections 14/16 from the lumen of the connector structure 18. In some embodiments, since the amount of expansion is very predictable and consistent, the wire components can be sized appropriately to compensate for this. Additionally, the bismuth alloy that may solidify outside of the connector 18 can server to form or function as strain relief just proximal and distal of the connector. In some embodiments, bismuth alloy that may solidify outside of the connector 18 may have a constant diameter, which can be beneficial for strain relief.

Alternatively, a sufficient amount of the bismuth alloy connector material can be applied to either one or both ends 24/26 of the proximal and distal guidewire sections 14/16, and either one or both ends 24/26 are inserted into a bonding position within the lumen within the connector tube 18 prior to solidification of the bismuth alloy connector material. The bismuth alloy connector material 30 can then be allowed to cool, solidify, and expand to provide a connection as discussed above.

Another alternative method can entail disposing the bismuth alloy connector material within the lumen of the connector structure 18 prior to insertion of the ends 24/26 into the lumen. In some such embodiments, the ends 24/26 can be inserted in a bonding position within the lumen prior to solidification of the bismuth alloy connector material. In some other such embodiments, the bismuth alloy connector material can be allowed to solidify, and is then reheated prior to or during the insertion of the ends 24/26 into a bonding position Within the lumen. Again, the bismuth alloy connector material 30 can then be allowed to cool, solidify, and expand to provide a connection as discussed above.

In most cases, a permanent connection (as opposed to a releasable connection) is made. However, due to the nature of the bismuth alloy connector material, the joint can be disconnected, or reworked by reheating the connector material and separating or reworking the components of the joint.

Once connected, the connector tube 18 and the proximal and distal guidewire sections 14/16 can be worked or formed to provide desired characteristics, such as shape or flexigility characteristics. For example, connector tube 18 and the proximal and distal guidewire sections 14/16 can be centerless ground to provide a smooth and uniform profile across the connection 20, and to straighten out small misalignments between the proximal and distal guidewire sections 14/16. Other portions of the guidewire 10 may be ground as well to provide the desired tapers and changes in diameter. For example, one or both of the proximal and distal guidewire sections 14/16 can be continuously tapered, can have a tapered section or a number or series of tapered sections of differing diameters, or can have a constant diameter. In some embodiments, the sections 14/16 are tapered or otherwise formed to have a geometry that decreases in cross sectional area toward the distal end thereof. If tapered, the sections 14/16 can include a uniform or a non-uniform transition of the sections, depending on the transition characteristics desired. For example, one or both of the sections 14/16 may be linearly tapered, tapered in a curvilinear fashion, or tapered in a step-wise fashion. The angle of any such tapers can vary, depending upon the desired flexibility characteristics. The length of the taper may be selected to obtain a more (longer length) or less (shorter length) gradual transition in stiffness. The centerless grinding technique may utilize an indexing system employing sensors (e.g., optical/reflective, magnetic) to avoid excessive grinding of the connection 20. In some embodiments, the presence of dissimilar materials in the construction can influence the grinding technique and tooling used to accomplish uniform material removal, create smooth transitions, and successfully bridge across adjacent components. In addition, the centerless grinding technique may utilize a CBN or diamond abrasive grinding wheel that is well shaped and dressed to avoid grabbing the connector 20 during the grinding process.

Once finally ground or otherwise worked or shaped, in some embodiments, a flexible coil (such as a coil tip) and/or a polymer jacket (such as a polymer tip) (optionally covering connection 20) or combination thereof, and other such structure, such as radiopaque markers, safety and/or shaping ribbons (coiled or uncoiled), and the like, may be placed on the guidewire 10, for example, adjacent the distal portion. Some examples of guidewire constructions, for example tip constructions, are disclosed in U.S. patent application Ser. No. 10/068,992 filed Feb. 28, 2002, entitled “Composite Guidewire”, which is incorporated herein by reference. Additionally, in some embodiments, a coating, for example a lubricious (e.g., hydrophylic) or other type of coating may be applied to all or portions of the guidewire. Different coatings can be applied to different sections of the guidewire. Some examples of such coatings and materials and methods used to create such coatings can be found in U.S. Pat. Nos. 6,139,510 and 5,772,609, which are incorporated herein by reference.

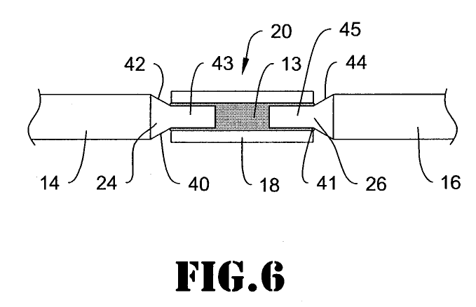

Refer now to FIG. 6, which illustrates another example embodiment similar to the embodiment of FIG. 3, except that the connection 20 between the proximal guidewire section 14 and the distal guidewire section 16 uses a butt type joint 13, but wherein the joined ends 24/26 are spaced from one another. Additionally, in the embodiment of FIG. 6, the joined ends 24/26 include reduced diameter portions 40 and 41. The reduced diameter portion 40 includes tapering portion 42 and constant diameter portion 43. Reduced diameter portion 41 includes tapering portion 44 and constant diameter portion 45. The constant diameter portions 43 and 45 are configured to fit within the connector 18, and are attached to the connector 18 using a bismuth fusible alloy material, as discussed above. Some additional examples of structures and materials that can be used in medical device constructions are also disclosed in a U.S. Patent Application entitled “ELONGATED INTRACORPORAL MEDICAL DEVICE” (Attorney docket no. 1001.1673101) filed on even date herewith, which is incorporated herein by reference.

It should be understood that this disclosure is, in many respects, only illustrative. Changes may be made in details, particularly in matters of shape, size, and arrangement of steps without exceeding the scope of the invention. For example, alternative structure can be used in connecting the proximal and distal sections of medical devices. Additionally, alternative tip constructions including a flexible coil tip, a polymer jacket tip, a tips including a coiled safety/shaping wire, or combination thereof, and other such structure may be placed on the medical device. The invention's scope is, of course, defined in the language in the claims.

What is claimed is:

1. A medical device, comprising:
an elongated section having an end;
a structural member defining an opening therein, wherein the end of the elongated section extends into the opening; and
a bismuth alloy connector material disposed within the opening, the connector material configured to expand when solidified to exert a compressive force within the opening in the structural member.

2. The medical device of claim 1, wherein the bismuth alloy connector material is solidified and has expanded to exert a compressive force on the structural member and the end of the elongated section to provide a mechanical interlock between the structural member and the elongated section.

3. The medical device of claim 1, wherein the structural member defines a second opening therein, and the medical device further includes a second elongated section having an end, wherein the end of the second elongated section extends into the second opening; and
a bismuth alloy connector material disposed within the second opening, the connector material configured to expand when solidified to exert a compressive force within the structural member.

4. The medical device of claim 3, wherein the first opening and the second opening are openings at the opposite ends of a common lumen defined in the structural member.

5. The medical device of claim 1, wherein the bismuth alloy connector material has a eutectic melting point in the range of 200° C. or below.

6. The medical device of claim 1, wherein the bismuth alloy connector material has a eutectic melting point in the range of 150° C. or below.

7. The medical device of claim 1, wherein the bismuth alloy connector material comprises in the range of about 4 to about 80 wt. % bismuth.

8. The medical device of claim 1, wherein the bismuth alloy connector material comprises bismuth and an additional alloying element selected from tin, indium, or mixtures thereof.

9. The medical device of claim 1, wherein the bismuth alloy connector material comprises in the range of about 35 to about 45 wt. % bismuth, and in the range of about 55 to about 65 wt. % tin.

10. The medical device of claim 1, wherein the bismuth alloy connector material comprises in the range of about 53 to about 63 wt. % bismuth, and in the range of about 37 to about 47 wt. % tin.

11. The medical device of claim 1, wherein the bismuth alloy connector material comprises a eutectic bismuth-tin alloy.

12. The medical device of claim 1, wherein the bismuth alloy connector material comprises in the range of about 2 to about 10 wt. % bismuth, and in the range of about 90 to about 98 wt. % indium.

13. The medical device of claim 1, wherein the bismuth alloy connector material comprises in the range of about 62 to about 72 wt. % bismuth, and in the range of about 28 to about 38 wt. % indium.

14. The medical device of claim 1, wherein the bismuth alloy connector material comprises in the range of about 29 to about 39 wt. % bismuth, and in the range of about 61 to about 71 wt. % indium.

15. The medical device of claim 1, wherein the bismuth alloy connector material comprises a eutectic bismuth-indium alloy.

16. The medical device of claim 1, wherein the bismuth alloy connector material comprises in the range of about 53 to about 63 wt. % bismuth, in the range of about 20 to about 30 wt. % indium, and in the range of about 12 to about 22 wt. % tin.

17. The medical device of claim 1, wherein the bismuth alloy connector material comprises a eutectic bismuth-indium-tin alloy.

18. The medical device of claim 1, wherein the structural member comprises a metal or metal alloy.

19. The medical device of claim 1, wherein the medical device comprises a guidewire.

20. A medical device, comprising:
a proximal section having a distal end;
a distal section having a proximal end;
a connector member defining a lumen having a first opening and a second opening, wherein the distal end of the proximal section extends into the lumen through the first opening and the proximal end of the distal section extends into the lumen through the second opening; and
a bismuth alloy connector material disposed within the lumen, the connector material configured to expand when cured to provide when solidified to exert compressive forces within the connector member.

21. The medical device of claim 20, wherein the bismuth alloy connector material is solidified and has expanded to exert a compressive force on the connector member and the distal end of the proximal section and the proximal end of the distal section to provide a mechanical interlock between the connector member and the ends of the elongated sections.

22. A medical device as in claim 20, wherein the proximal section has a first flexibility and the distal section has a second flexibility, and wherein the distal end of the proximal section and the proximal end of the distal section overlap to define a region that blends the first flexibility with the second flexibility.

23. A medical device as in claim 20, wherein the distal end of the proximal section has a reduced size, and the proximal end of the distal section has a reduced size.

24. A medical device as in claim 23, wherein the reduced size portions have a uniform profile.

25. A medical device as in claim 23, wherein the reduced size portions have a taper.

26. A medical device as in claim 23, wherein the reduced size portions have an interlocking shape.

27. A medical device as in claim 20, wherein the distal end of the proximal section and the proximal end of the distal section are joined to define a butt joint within the connector member.

28. A medical device as in claim 20, wherein the distal end of the proximal section defines a tapered portion and the proximal end of the distal section defines a tapered portion, and the tapered portions at least partially overlap each other.

29. A medical device as in claim 20, wherein the proximal section comprises a metal or metal alloy.

30. A medical device as in claim 29, wherein the metal or metal alloy comprises stainless steel, nickel-titanium alloy, nickel-chromium alloy, nickel-chromium-iron alloy, cobalt alloy, or combinations thereof.

31. A medical device as in claim 29, wherein the proximal section comprises stainless steel.

32. A medical device as in claim 20, wherein the medical device further includes an outer structure disposed about at least a portion of the distal section.

33. A medical device of claim 20, wherein the connector structure comprises a metal or a metal alloy.

34. A medical device of claim 33, wherein the metal or a metal alloy comprises stainless steel, nickel-titanium alloy, nickel-chromium alloy, nickel-chromium-iron alloy, cobalt alloy, nickel, or combinations thereof.

35. A medical device as in claim 34, wherein the connector comprises a nickel-titanium alloy.

36. A medical device of claim 20, wherein the connector comprises a polymer or a metal-polymer composite.

37. A medical device as in claim 20, wherein the connector structure comprises a tubular member disposed about the distal end of the proximal section and the proximal end of the distal section.

38. A medical device as in claim 20, wherein the distal section comprises a metal or metal alloy.

39. A medical device as in claim 38, wherein the metal or metal alloy comprises stainless steel, nickel-titanium alloy, nickel-chromium alloy, nickel-chromium-iron alloy, cobalt alloy, or combinations thereof.

40. A medical device as in claim 38, wherein the distal section comprises a nickel-titanium alloy.

41. A guidewire, comprising:
a proximal section having a distal end;
a distal section having a proximal end; and
a connector structure disposed adjacent the distal end of the proximal section and the proximal end of the distal section; and
means for exerting a compressive force on the connector structure and the ends of the proximal and distal sections to provide a mechanical interlock between the connector structure and proximal and distal sections.

42. A method of manufacturing a structure for use in a medical device, the method comprising:
providing an elongated section having an end;
providing a structural member defining an opening therein;
disposing the end of the elongated section into the opening;
disposing a bismuth alloy connector material within the opening; and
allowing the bismuth alloy connector material to solidify and expand to exert a compressive force within the opening in the structural member.

43. The method of claim 42, wherein the solidified and expanded bismuth alloy connector material exerts a compressive force on the structural member and the end of the elongated section to provide a mechanical interlock between the structural member and the elongated section.

44. The method of claim 42, wherein the structural member further defines a second opening therein, and further including:
providing a second elongated section having an end;
disposing the end of the second elongated section into the second opening;
disposing a bismuth alloy connector material within the second opening; and
allowing the bismuth alloy connector material to solidify and expand to exert a compressive force within the second opening in the structural member.

45. The method of claim 42, wherein the first opening and the second opening are openings at the opposite ends of a common lumen defined in the structural member.

46. The method of claim 42, wherein the bismuth alloy connector material has a eutectic melting point in the range of 200 C or below.

47. The method of claim 42, wherein the bismuth alloy connector material has a eutectic melting point in the range of 150 C or below.

48. The method of claim 42, wherein the bismuth alloy connector material comprises in the range of about 4 to about 80 wt. % bismuth.

49. The method of claim 42, wherein the bismuth alloy connector material comprises bismuth and an additional alloying element selected from tin, and indium.

50. The method of claim 42, wherein the bismuth alloy connector material comprises a bismuth-tin alloy.

51. The method of claim 42, wherein the bismuth alloy connector material comprises a eutectic bismuth-tin alloy.

52. The method of claim 42, wherein the bismuth alloy connector material comprises a bismuth-indium alloy.

53. The method of claim 42, wherein the bismuth alloy connector material comprises a eutectic bismuth-indium alloy.
